(19) United States
(12) Patent Application Publication
(10) Pub. No.: US 2022/0180966 A1
(43) Pub. Date: Jun. 9, 2022

(54) USE OF GENE EXPRESSION DATA AND GENE SIGNALING NETWORKS ALONG WITH GENE EDITING TO DETERMINE WHICH VARIANTS HARM GENE FUNCTION

(71) Applicants: THEMBA INC., SAN CARLOS, CA (US); MYOME, INC., PALO ALTO, CA (US)

(72) Inventors: MATTHEW RABINOWITZ, SAN FRANCISCO, CA (US); OREN SCHAEDEL, SAN FRANCISCO, CA (US)

(73) Assignees: THEMBA INC., SAN CARLOS, CA (US); MYOME, INC., PALO ALTO, CA (US)

(21) Appl. No.: 17/599,343

(22) PCT Filed: Mar. 30, 2020
(86) PCT No.: PCT/US20/25670

§ 371 Date: Sep. 28, 2021

Related U.S. Application Data
(60) Provisional application No. 62/825,037, filed on Mar. 28, 2019.
(60) Provisional application No. 62/923,407, filed on Oct. 18, 2019.

(51) Int. Cl. G16B 20/20; G16B 25/10; G16B 40/20

(52) U.S. Cl. CPC G16B 20/20; G16B 40/20; G16B 25/10

(57) ABSTRACT

Provided are methods of determining the pathogenicity of a genetic variant, the method comprising: selecting a gene of interest; identifying a network of genes or gene variants that are up-regulated, down-regulated, or co-expressed with the gene of interest, and/or that interact directly or indirectly with the gene of interest; and determining the pathogenicity of the gene of interest based on the presence, absence, and/or expression levels of the network of genes. Also provided are methods of constructing a pathogenicity classifier for a genetic variant, the method comprising training a pathogenicity classifier.

### CROSS REFERENCE TO RELATED APPLICATIONS

This application claims the benefit of U.S. Provisional Application No. 62/825,037, filed on Mar. 28, 2019, and U.S. Provisional Application No. 62/923,407, filed on Oct. 18, 2019, each of which are incorporated herein by reference in their entirety.

### FIELD

Described are methods for identifying pathogenic genes and gene networks.

### BACKGROUND

A variety of tools exist to determine whether or not a mutation in a gene is causing a disease; however, these tools are still severely lacking. A large number of mutations in a person's genome (e.g., in a whole exome or whole genome sequence) are classified as “likely benign,” “likely pathogenic,” or “unknown significance,” and the available tools do not provide enough information to determine whether the variant effect the protein in a significant way to cause a disease or another phenotype. Thus, there is a need in the art for an improved method of identifying pathogenic genetic variants.

### SUMMARY

Provided are methods of determining the pathogenicity of a genetic variant. In some aspects, the methods comprise receiving, by a processing device, a first training dataset that includes expression data for a gene of interest and one or more other genes that are up-regulated, down-regulated, or co-expressed with the gene of interest, and/or that interact directly or indirectly with the gene of interest or genes that the gene of interest interacts directly or indirectly with. Some aspects comprise receiving a second dataset that includes phenotypic and/or genotypic data associated with the presence, absence, and/or expression levels of the gene of interest and/or the other genes. Some aspects comprise training a model on the first and second training datasets to identify a network of genes associated with phenotypic outcomes. Some aspects comprise scoring the pathogenicity of the gene of interest based on the network of genes associated with phenotypic outcomes.

In some aspects, the methods further comprising receiving a list of genes from a third dataset that includes data associated with known gene-gene, gene-protein and/or protein—protein interaction networks for the gene of interest and/or the other genes, and training the model using the first, second, and third datasets to identify the network of genes associated with phenotypic outcomes.

In some aspects, the second dataset comprises phenotypic data associated with benign other genes and pathogenic other genes.

In some aspects, the training comprises one or both of (i) normalizing and standardizing expression levels of the gene of interest and/or the other genes, and (ii) resampling data in the training samples.

In some aspects, one or more of SVM, linear regression, logistic regression, random forest, naive bayes, and adaboost are used to identify the network of genes or gene variants associated with the phenotypic outcomes.

In some aspects, the first dataset and/or the second dataset each independently comprises data from a plurality of datasets.

In some aspects, the first and/or second training datasets independently include data from one or more tissues selected from brain, heart, lung, kidney, liver, muscle, bone, stomach, intestines, esophagus, and skin tissue; and/or one or more of a biological fluids selected from urine, blood, plasma, serum, saliva, semen, sputum, cerebral spinal fluid, mucus, sweat, vitreous liquid, and milk. In some aspects, a test set may include different sets of tissues and/or biological fluids from the tissues and/or biological fluids used for training.

In some aspects, the gene of interest is associated with an autosomal dominant condition. In some aspects, the gene of interest is associated with an autosomal recessive condition. In some aspects, the gene of interest is associated with a risk for a non-Mendelian condition.

In some aspects, the first dataset is comprised of mRNA and/or protein expression data associated with the gene of interest and/or the other genes or gene variants.

In some aspects, the gene of interest is a genetic variant of interest.

Also provided are methods of constructing a pathogenicity classifier for a genetic variant, the method comprising training a pathogenicity classifier on a processing device, wherein the method comprises using as input: (i) a first training dataset that includes expression data for a gene of interest and one or more other genes that are up-regulated, down-regulated, or co-expressed with the gene of interest, and/or that interact directly or indirectly with the gene of interest or with genes that the gene of interest directly or indirectly interacts with; and (ii) a second training dataset that includes phenotypic and/or genotypic data associated with the presence, absence, and/or expression levels of the other genes; and wherein the method comprises identifying a network of genes that are up-regulated, down-regulated, or co-expressed with the gene of interest, and/or that interact directly or indirectly with the gene of interest or with genes that the gene of interest directly or indirectly interacts with.

Some aspects comprise using as input a third training sample from a third dataset that includes data associated with known gene-gene or gene-protein interactions for the gene of interest and/or the other genes.

Also provided are methods of determining the pathogenicity of a genetic variant, the method comprising: performing genotyping, and establishing that there is a variant of unknown significance; measuring expression levels in one or more samples from one or more subjects with the variant, and a trained model; and determining the pathogenicity of the gene of interest based on the presence, absence, and/or expression levels of the network of genes. In some aspects, the genotyping comprises performing whole genome sequencing, whole exome sequencing or target panel sequencing.

Some aspects comprise obtaining mRNA and/or protein expression data associated with the gene of interest and/or the other genes.

In some aspects, the first dataset comprises data from a genetic screen that comprises (i) perturbing the DNA of one or more cells and inserting a variant of known significance (benign or pathogenic), and (ii) validating that that the genetic variant of known significance is classifying correctly using measured expression levels.

In some aspects, the second dataset comprises data from a genetic screen that comprises (i) perturbing the DNA of one or more cells, and (ii) determining the phenotype associated with the one or more perturbed cells.

In some aspects, the perturbing is conducted with CRISPR.

In some aspects, the measured expression levels comprise levels of mRNA of a network of genes identified in the classification model.

Also provided are methods comprising: identifying a genetic variant in a sample taken from a subject; obtaining gene expression data from the subject; applying the trained model to the gene expression data; and determining whether the genetic variant is pathogenic. Some aspects comprise obtaining a plurality of tissue and/or biological fluid samples from the subject and performing gene expression analysis on the plurality of tissue and/or biological fluid samples. Some aspects comprise obtaining gene expression data from one or more tissue and/or biological samples from one or more additional subjects; combining the gene expression data from the subject and the one or more additional subjects; applying the trained model to the combined data; and determining whether the genetic variant is pathogenic. In some aspects, the gene expression data comprises mRNA and/or protein expression data.

### BRIEF DESCRIPTION OF DRAWINGS

FIG. 1A sets forth a diagram demonstrating gene network interactions; FIG. 1B sets forth a diagram, and a protocol for identifying a gene network linked with a phenotype of interest.

FIG. 2 sets forth CSV data files used in Example 1.

FIG. 3 sets forth data and graphical representations associated with identifying gene networks using the publicly available tools String v11 (3A) and Genemania (3B).

FIG. 4 sets forth graphical representations associated with data obtained by using different gene ensembles as features (X axis) while using different binary classifiers: (A) SVM, (B): Logistic Regression, (C) Random Forest.

FIG. 5 sets forth graphical representations associated with oversampled benign samples obtained by using different gene ensembles as features (X—axis) while using different binary classifiers: (A) SVM, (B): Logistic Regression, (C) Random Forest.

FIG. 6 is a block diagram of an example computing device.

### DETAILED DESCRIPTION

Technical and scientific terms used herein have the meanings commonly understood by one of ordinary skill in the art to which the present invention pertains, unless otherwise defined. Materials to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “gene” relates to stretches of DNA or RNA that encode a polypeptide or that play a functional role in an organism. A gene can be a wild-type gene, or a variant or mutation of the wild-type gene. A “gene of interest” refers to a gene, or a variant of a gene, that may or may not be causative of a phenotype of interest. An “other gene” refers to a gene other than the gene of interest. A “gene network” includes one or more other genes or gene variants that collectively are associated with a phenotype of interest. The gene network may or may not include the gene of interest.

“Expression” refers to the process by which a polynucleotide is transcribed from a DNA template (such as into a mRNA or other RNA transcript) and/or the process by which a transcribed mRNA is subsequently translated into peptides, polypeptides, or proteins. Expression of a gene encompasses not only cellular gene expression, but also the transcription and translation of nucleic acid(s) in cloning systems and in any other context. Where a nucleic acid sequence encodes a peptide, polypeptide, or protein, gene expression relates to the production of the nucleic acid (e.g., DNA or RNA, such as mRNA) and/or the peptide, polypeptide, or protein. Thus, “expression levels” can refer to an amount of a nucleic acid (e.g. mRNA) or protein in a sample.

Described are novel and unpredictable methods for using gene expression databases to determine whether or not a particular genetic variant is implicated in a disease, for instance by evaluating the presence, absence, and/or expression levels of upstream, midstream, and/or downstream genes, and/or genes that interact with the genetic variant of interest. Some aspects involve checking the “downstream” genes in the signaling pathway that are regulated by the gene of interest and collecting statistically significant data to determine if the gene still regulates of the downstream genes. The downstream expression data may be analyzed in combination with data of the gene of interest, data from other “midstream” genes that regulate the same downstream genes that are regulated by the gene of interest, and data from “upstream” genes that regulate the gene of interest.

Also described are methods of using gene expression as a functional assay in a laboratory measurement to determine whether a gene of interest causes a particular phenotype. For instance, a model can be created to describe the gene expression and the gene signaling relationships in cells that do not have a known deleterious mutation. Then, using a genetic perturbation technique, such as with CRISPR/Cas9, one or more mutations are introduced to a set of cells. The expression of RNA or proteins in the cells can then be analyzed to determine whether the mutations cause a significant change in the gene expression or gene signaling relationships.

Without being bound by theory, it is believed that some methods described here are associated with one or more of the following advantages: (i) they are not dependent on the particular choice of cells and can be used in multiple different tissue types so long as the models are consistent with those tissue types and the relevant organism, which would typically be humans, (ii) they do not require additional experimentation to measure the functional phenotype of a particular gene in the lab, which is not easily scalable and not time and cost effective; but rather performs standardized measurements of gene expression when necessary for a particular sample and assess the phenotype in silico, and/or (iii) they are a more reliable and encompassing method for measuring functional phenotypes of mutations which will become more powerful over time as gene expression databases grow.

### Gene Selection

The gene of interest can be identified by any means known in the art. For instance, the gene of interest can be selected based on a subject's personal genome. In some aspects, the gene of interest is a genetic variant of unknown pathogenicity, e.g., because the gene is not statistically significantly associated with an observed phenotype. In some aspects, the gene is associated with an autosomal dominant condition. In other aspects, the gene is associated with an autosomal recessive condition. In some aspects, the gene of interest is associated with a risk for a non-Mendelian condition.

Genes or gene variants other than the gene of interest can be identified by comparing a first dataset (e.g., having genotype-tissue expression data) to a second dataset (e.g., having genotype-phenotype data). In some aspects, the one or more other genes or gene variants are up-regulated, down-regulated, and/or co-expressed with the gene of interest. In some aspects, the one or more other genes or gene variants interact directly or indirectly with the gene of interest.

In some aspects, transcripts (model features) are selected based in part on known or predicted protein and genetic interactions, pathways, co-expression, co-localization and protein domain similarity, e.g., as indicated by GeneMania or String version 11 and a large selection of other gene expression databases involving RNA or protein or other measures of gene interaction or expression. In some aspects, transcripts are removed from the feature set using standard methods of regularization and muticollinearity correction.

Datasets for identifying genes of interest and/or other genes or gene variants can be obtained by any means known in the art. For instance, genes or gene variants in a network of genes or gene variants can be identified in a first dataset. The first dataset can include expression data for genes of interest and one or more other genes or gene variants. The one or more other genes or genes variants can be up-regulated, down-regulated, or co-expressed with the gene of interest. The one or more other genes or gene variants can interact directly or indirectly with the gene of interest. The gene of interest can regulate the other genes or gene variants, or vice versa. In some aspects, one or more other genes or gene variants interact directly or indirectly with the gene of interest or genes that the gene of interest interacts directly or indirectly with. In some aspects, the one or more other genes or gene variants is upstream, midstream, and/or downstream as compared to the gene of interest.

Other genes or gene variants can be identified based on known or determined gene pathways. Such pathways can be identified using publicly available databases, such as Reactome, KEGG Pathway Database, or the many different pathway databases described by the Human Sciences Library System (see world wide web at hsls.pitt. edu/obrc/index.php?page=signaling_pathways, under the category: HSLS Home>Molecular Biology>OBRC: Online Bioinformatics Resources Collection>Signaling Pathways). One can measure, or extract data from online databases on the expression level of a gene of interest and/or of downstream genes and/or of upstream genes, for one or more subjects that have the gene of interest. Additional exemplary databases include Expression Atlas (ebi.ac.uk/gxa/home); the Gene Expression Omnibus (GEO) (ncbi.nlm.nih.gov/geo/info/datasets.html); and Lifemap (discovery.lifemapsc.com/gene-expression-signals), which is linked to the human disease database Malacards (malacards.org/) (see doi. org/10.1093/nar/gkw1012).

In some aspects, the first dataset is compiled from genotype-tissue expression analysis, e.g., from one or more publicly available databases, such as the GTEx Project. In some aspects, the first dataset is compiled from one or more of whole exome sequencing (WES) data, whole genome sequence (WGS) data, and/or Illumina array data (e.g. from a SNP array). In some aspects the first dataset comprises data from a genetic screen, e.g., that comprises perturbing the DNA of one or more cells, and then determining other of genes or gene variants by sequencing RNA from the one or more perturbed cells. In some aspects, the first dataset comprises data from subjects that have tissues that have undergone RNA-seq.

A second dataset can be used to determine phenotypic characteristics associated with the gene of interest and/or other genes or gene variants. In some aspects, the second dataset is compiled based on data implicating genes or gene variants in disease. Thus, the second dataset can be used to classify one or more of the genes or gene variants as pathogenic, likely pathogenic, unknown pathogenicity, likely benign, or benign. In some aspects the second data set is compiled from one or more publicly available databases, such as Clinvar, Online Mendelian Inheritance in Man (OMIM) database, Leiden Open Variation Databse (LOVD), Human Gene Mutation Database (HGMD), ClinGen, dbVar, HmtVar, BRCA exchange, Instem.res.in, and/or cBioPortal. In some aspects the second dataset comprises data from a genetic screen, e.g., that comprises perturbing the DNA of one or more cells, and then determining the phenotype associated with the one or more perturbed cells. In some aspects, the second dataset comprises data from subjects that have tissues that have undergone RNA-seq.

The datasets can be compiled using data from one or more of a variety of tissues or body fluids. For instance, the first and/or second dataset can independently include data associated with brain tissue, heart tissue, lung tissue, kidney tissue, liver tissue, muscle tissue, bone tissue, stomach tissue, intestines tissue, esophagus tissue, and/or skin tissue, or any combination of such tissues. Additionally or alternatively, the datasets can include data associated with biological fluids, such as urine, blood, plasma, serum, saliva, semen, sputum, cerebral spinal fluid, mucus, sweat, vitreous liquid, and/or milk, or any combination of such fluids.

In some aspects the datasets are compiled using data from subjects having a particular condition or conditions, and/or a particular symptom or symptoms. Preferably, the datasets are compiled using samples from a plurality of subjects. In some aspects, the datasets are compiled using samples from a plurality of tissues and/or a plurality of biological fluids.

### Gene Network Identification

Some aspects comprise identifying a network of other genes that are up-regulated, down-regulated, or co-expressed with the gene of interest, and/or that interact directly or indirectly with the gene of interest, and/or that interact directly or indirectly with the gene of interest or genes that the gene of interest interacts directly or indirectly with; and determining the pathogenicity of the gene of interest based on the presence, absence, and/or expression levels of the network of genes or gene variants. The gene network can include, in addition to the gene of interest, 1, 2, 3, 4, 5, 6, 7, 8, 9, 10, 20, 30, 40, or 50 or more, such as 100 or more, genes.

A gene network can be identified from the other genes selected from a plurality of datasets. The gene network can be identified using machine learning (including supervised and/or unsupervised machine learning algorithms). In some aspects, the gene network can be identified by training a model (e.g., a classifier) on training samples from a first dataset (e.g., having genotype-tissue expression data) and a second dataset (e.g., having genotype-phenotype data). In some aspects, the training includes normalization (e.g., normalizing transcript expression levels of genes of interest and/or other genes or gene variants to expression levels of housekeeping genes) and/or standardization steps (e.g., via SVM to scale transcript abundance to zero mean).

A classification model can be developed from the other genes selected from the plurality of datasets. The classification model can be trained using machine learning (including supervised and/or unsupervised machine learning algorithms). In some aspects, the classification model is trained by training a classifier filtering training labels training samples from a first dataset (e.g., having genotype-tissue expression data) and a second dataset (e.g., having genotype-phenotype data). In some aspects, the training includes normalization (e.g., normalizing transcript expression levels of genes of interest and/or other genes or gene variants to expression levels of housekeeping genes) and/or standardization steps (e.g., via SVM to scale transcript abundance to zero mean).

In some aspects, the classification model can be trained using resampling techniques, such as oversampling or undersampling. For instance, pathogenic variants can be oversampled when using a test-train split method, and/or benign variants can be undersampled. Other techniques can be used to accommodate the difference in number of pathogenic vs benign variants in the database without changing the concept. Some aspects comprise using binning and/or bagging techniques to identify the gene network. In some aspects, parametric and/or non-parametric statistical tests are used to evaluate expression differences between pathogenic and non-pathogenic variants.

In some aspects, a classification model can be used to classify a gene of interest as being causative of a condition or phenotype. For instance, a binary classification system can be used. As an example, for autosomal dominant conditions, pathogenic (0/1 and 1/1) variants per allele per gene can be encoded as positives and pathogenic (0/0) variants and benign variants can be encoded as negatives. As a further example, for autosomal recessive conditions, only pathogenic (1/1) variants per allele per gene can be encoded as a positive and all other genotypes can be encoded as a negative. Classification can be performed using a host of techniques, for instance, linear regression, linear and nonlinear SVM, logistic regression, random forest, nave bayes, adaboost and/or other methods of combining different kinds of classifiers.

In some aspects, the predictive performance of the gene network or classification model is scored using an area under the curve (AUC) measurement. For instance, the AUC can be more than about 0.75, more than about 0.8, more than about 0.85, more than about 0.9, more than about 0.95, more than about 0.97, more than about 0.98, or more than about 0.99. The gene network and/or classifier can be optimized to maximize AUC, precision, and recall.

### Computing Significance of Effect of Mutation on Gene Expression

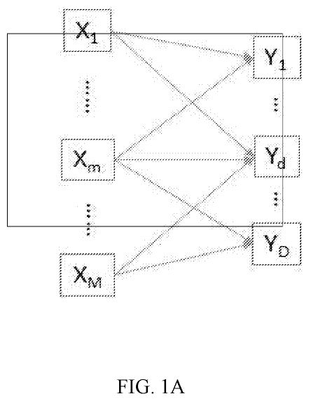

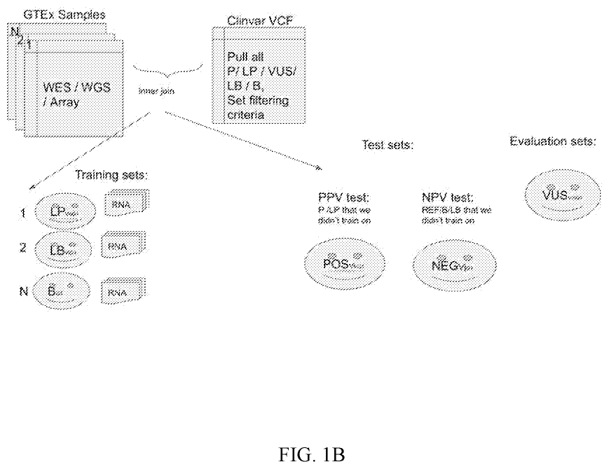

As already mentioned, described are aspects in which the impact a gene of interest is evaluated by considering the effect of the gene on other genes or gene variants. For instance, FIG. 1 sets forth an exemplary gene signaling network, involving a gene that has a mutation in the subject of interest. Let Xₘ be the random variable representing the RNA or protein expression level of the gene that carries a mutation in the subject. For convenience, notations like Xₘ interchangeably refer to the gene, and to the random variable representing the gene's expression level. Assume that gene Xₘ affects the expression level of D downstream genes Y₁ . . . Y_{D}. Assume M is the number of “midstream” genes, including the mutated gene, that affect one or more of the D downstream genes. The gene expression signal level of the M midstream genes are represented by X₁ . . . X_{M}.

In some aspects, one dataset for individuals with the mutated gene is compared to another dataset of individuals without the mutated gene, e.g., to check for systemic biases caused by the mutation. Such information in the signaling network can be used to improve the statistical significance of the comparison. For instance, suppose there are N subjects in the control dataset where gene m is not mutated. The set of expression levels of gene Xₘ for the N subjects can be represented by random variables Xₘ₁ . . . X_{mN} and measured values xₘ₁ . . . x_{mN}. In practice, to facilitate combining databases or nonlinear models, the measured expression levels in a dataset can be normalized, for example, by removing the mean bias and normalizing the absolute signal level for each gene. One possible normalization across subjects in a database can be set forth as:

$${x}_{mn} \to \frac{{x}_{mn} - \frac{1}{N} {\sum }_{i = 1}^{N} {x}_{mi}}{\frac{1}{N} {\sum }_{i = 1}^{N} \vert {x}_{mi} \vert}$$

Assuming that all expression levels have gone through some form of normalization, the notation is not changed. For simplicity, start with one downstream gene Y_{d} which is affected by all M midstream genes and has expression levels y_{d1} . . . y_{dN}. For now, drop the d subscript and refer to downstream gene Y with expression levels y₁ . . . y_{N}.

One model for computing significance levels accounting for the network can be represented in matrix format. For the set of N subjects who do not have the variant, the downstream gene can be represented as:

$$y = x β + γ ;$$

$$x = μ + ∝ ;$$

$$y , \\ γ ∈ {ℝ}^{N \times 1} ;$$

$$β ∈ {ℝ}^{( M + 1 ) \times 1} ;$$

$$x ∈ {ℝ}^{N \times ( M + 1 )}$$

$$where$$

$$x = [ \begin{matrix}1 & {x}_{11} & ⋯ & {x}_{M 1} \\ ⋮ & ⋮ & & ⋮ \\ 1 & {x}_{1 N} & ⋯ & {x}_{MN}\end{matrix} ] , \\ y = [ \begin{matrix}{y}_{1} \\ ⋮ \\ {y}_{N}\end{matrix} ] , \\ β = [ \begin{matrix}{β}_{0} \\ ⋮ \\ {β}_{M}\end{matrix} ] , \\ γ = [ \begin{matrix}{y}_{1} \\ ⋮ \\ {y}_{N}\end{matrix} ] , \\ ∝ = [ \begin{matrix}1 & {∝}_{11} & ⋯ & {∝}_{M 1} \\ ⋮ & ⋮ & & ⋮ \\ 1 & {∝}_{1 N} & ⋯ & {∝}_{MN}\end{matrix} ]$$

and where γₙ, n=1 N represent measurement error or noise and are all i.i.d (independent identically distributed) random variables with a normal distribution γₙ˜N (0, σ_{γ}²). For those subjects that have the variant of interest, expression levels may be represented with a model as follows:

$${y}_{v} = {x}_{v} {β}_{v} + {γ}_{v} ;$$

$${x}_{v} = {μ}_{v} + {α}_{v} ;$$

$${y}_{v} , \\ {γ}_{v} ∈ {ℝ}^{P \times 1} ;$$

$${β}_{v} ∈ {ℝ}^{( M + 1 ) \times 1} ;$$

$$x , \\ {α}_{v} ∈ {ℝ}^{P \times ( M + 1 )}$$

$$where$$

$$x = [ \begin{matrix}1 & {{x}_{v}}_{{ }_{11}} & ⋯ & {x}_{{v}_{M 1}} \\ ⋮ & ⋮ & & ⋮ \\ 1 & {x}_{{v}_{1 P}} & ⋯ & {x}_{{v}_{MP}}\end{matrix} ] , \\ y = [ \begin{matrix}{y}_{{v}_{1}} \\ ⋮ \\ {y}_{{v}_{P}}\end{matrix} ] , \\ {β}_{v} = [ \begin{matrix}{β}_{{v}_{0}} \\ ⋮ \\ {β}_{{v}_{M}}\end{matrix} ] , \\ {γ}_{v} = [ \begin{matrix}{y}_{{v}_{1}} \\ ⋮ \\ {y}_{{v}_{P}}\end{matrix} ] , \\ {∝}_{v} = [ \begin{matrix}1 & {∝}_{{v}_{11}} & ⋯ & {∝}_{{v}_{M 1}} \\ ⋮ & ⋮ & & ⋮ \\ 1 & {∝}_{{v}_{1 P}} & ⋯ & {∝}_{{v}_{MP}}\end{matrix} ]$$

where γᵥ_{ₙ}˜N(0, σ_{γ}_{ᵥ}²) and ∝ᵥ_{ₙₚ}˜N(0, σ_{∝}_{ᵥ}²). The possibility of model error is explicitly included here, where the level of the midstream genes x might be affected by various factors that would cause them to deviate from the expected value μ. The downstream genes are assumed to be affected by the undeviated value x. The null hypothesis H₀ is that the model for the subjects with the variants is the same as that for the unaffected subjects, namely βᵥ=β. Many approaches can be used to generate significance values to test whether H₀ is true, or whether mutation has significantly affected gene expression. One such approach is described below, but the method extends to other approaches, such as selecting one hypothesis from many using a maximum-likelihood approach, rather than using a single hypothesis rejection test.

A model can be built based on the data of non-mutated subjects. The least squares estimate of the regression parameters and the model for yᵥ can be given by:

ŷ=x\hat{β};ŷᵥ=xᵥ\hat{β}

Note that there are many other techniques for estimating the model parameters \hat{β} and ŷᵥ. For example, if one has a good analytical or empirical model for var(yᵥ)=Cᵥ one can pre-multiply by the whitening function

$${{C}_{v}}^{- \frac{1}{2}} ,$$

as will be discussed later. Other methods include using restriction functions to increase constraints on the regression problem if it is under-determined due to too many possible genes, or too little patient data. Restriction functions on the regression parameters could include L₂ norm as used in Ridge Regression, or L₁ norm as used in the LASSO Regression. One can also capture nonlinear interactions among the genes which may, for example, involve additional parameters in β applied to additional columns of x describing nonlinear interactions among the genes. One can also also use models that are nonlinear in β such as Neural Networks, including Deep Learning Neural Networks, or Support Vector Machines. Some of these methods have been described in Rabinowitz M, et al, 2006 (Accurate prediction of HIV-1 drug response from the reverse transcriptase and protease amino acid sequences using sparse models created by convex optimization. Bioinformatics 2006;22:541-549), which is herein incorporated by reference in its entirety.

Assuming for now the least squares approach, the error is considered in the prediction:

e=ŷ−y;eᵥ=ŷᵥ−yᵥ

The expected value of eᵥeᵥ^{T} can be computed as follows:

$$E \{ {e}_{v} {{e}_{v}}^{T} \} = E \{ ( {\hat{y}}_{v} - {y}_{v} ) {( {\hat{y}}_{v} - {y}_{v} )}^{T} \} = E \{ ( {x}_{v} \hat{β} - {x}_{v} β - {γ}_{v} ) {( … )}^{T} \} = E \{ ( ( {μ}_{v} + {α}_{v} ) {( {x}^{T} x )}^{- 1} {x}^{T} ( x β + γ ) - ( {μ}_{v} + {α}_{v} ) β - {γ}_{v} ) {( … )}^{T} \} = E \{ ( ( {μ}_{v} + {α}_{v} ) ( β + {( {x}^{T} x )}^{- 1} {x}^{T} γ ) - ( {μ}_{v} + {α}_{v} ) β - {γ}_{v} ) {( … )}^{T} \} = E \{ ( {{μ}_{v} ( {x}^{T} x )}^{- 1} {x}^{T} γ + {{α}_{v} ( {x}^{T} x )}^{- 1} {x}^{T} γ - {γ}_{v} ) {( {{μ}_{v} ( {x}^{T} x )}^{- 1} {x}^{T} γ + {{α}_{v} ( {x}^{T} x )}^{- 1} {x}^{T} γ - {γ}_{v} )}^{T} \}$$

Effects of modeling error captured by x=μ+∝ can be incorporated by performing first order expansion of ((μ+∝)^{T}(μ+∝))⁻¹ in ∝. However, here it is assumed that enough varied data is used for training on non-affected subjects and the model is accurate enough that the modelling error effect on x is negligible, hence ∝=0. It can be shown that the expectation reduces six terms out of the subsequent expression to 0 except for these three:

E{eᵥeᵥ^{T}}=E{μᵥ(x^{T}x)^{31 1}x^{T}γγ^{T}x(x^{T}x)⁻¹μᵥ^{T}+∝ᵥ(x^{T}x)⁻¹x^{T}γγ^{T}x(x^{T}x)⁻¹∝ᵥ^{T}+γᵥγᵥ^{t}}

Since the noise γ and γᵥ are i.i.d, the first term reduces to σ_{γ}²μᵥ(x^{T}x)⁻¹μᵥ^{T} and the third to σ_{γ}_{ᵥ}²I where I ϵ [symbol] ^{P×P} is the identity matrix. The second term involves both γ and ∝ᵥ. Since these noise terms are i.i.d and independent of one another:

$$E \{ {∝}_{v} {( {x}^{T} x )}^{- 1} {x}^{T} {γγ}^{T} {x ( {x}^{T} x )}^{- 1} {{∝}_{v}}^{T} \} = {{σ}_{γ}}^{2} E \{ {∝}_{v} {( {x}^{T} x )}^{- 1} {{∝}_{v}}^{T} \} = {{σ}_{γ}}^{2} {{σ}_{{∝}_{v}}}^{2} {\sum }_{i = 1 … M + 1} {Diag}_{i} ( {( {x}^{T} x )}^{- 1} ) I$$

where Diagᵢ denotes the i^{th} diagonal element of the matrix. So, the estimate can be written as

E{eᵥeᵥ^{T}}=Cₑ_{ᵥ}=σ_{γ}²μᵥ(x^{T}x)⁻¹μᵥ^{T}+σ_{γ}²σ_{∝}_{ᵥ}²Σ_{i=1 . . . M+1}Diagᵢ((x^{T}x)⁻¹)I+σ_{γ}_{ᵥ}²I

Many test statistics can be derived based on this. For example, the Sum-Squared Error SSEᵥ is eᵥ^{T}eᵥ. The expected value of the SSE can be generated from the sum of the diagonal terms of Cₑ_{ᵥ}:

$$E \{ {SSE}_{v} \} = E \{ {{e}_{v}}^{T} {e}_{v} \} = {\sum }_{i = 1 … P} {Diag}_{i} ( {C}_{{e}_{v}} ) = {{σ}_{γ}}^{2} {\sum }_{i = 1 … P} {{μ}_{{v}_{i}} ( {x}^{T} x )}^{- 1} {{μ}_{{v}_{i}}}^{T} + {{σ}_{γ}}^{2} {{σ}_{{∝}_{v}}}^{2} P {\sum }_{i = 1 … M + 1} {Diag}_{i} ( {( {x}^{T} x )}^{- 1} ) + {{σ}_{{γ}_{v}}}^{2} P$$

Where P as a reminder is the number of subjects with the variant. As in this example \hat{β} was not generated from the subjects with the variant, and degrees of freedom are not lost by fitting the data to generate SSEᵥ. Consequently, the Mean-Squared Error which is Sum-Squared Error divided by degrees of freedom is

$${MSE}_{v} = \frac{{SSE}_{v}}{P} :$$

$$E \{ {MSE}_{v} \} = \frac{1}{P} ( {{σ}_{γ}}^{2} {\sum }_{i = 1 … P} {{μ}_{{v}_{i}} ( {x}^{T} x )}^{- 1} {{μ}_{{v}_{i}}}^{T} + {{σ}_{γ}}^{2} {{σ}_{{∝}_{v}}}^{2} {\sum }_{i = 1 … M + 1} {Diag}_{i} ( {( {x}^{T} x )}^{- 1} ) + {{σ}_{{γ}_{v}}}^{2} )$$

Now, noise variance terms can be estimated. This can be done using empirical data or estimates to make the test statistic conservative. For simplicity, consider one case where the noise model does not change between training and test data: σ_{γ}_{ᵥ}=σ_{γ}, σ_{∝}=0, xᵥ=μᵥ, in which case

$$E \{ {MSE}_{v} \} = \frac{{{σ}_{γ}}^{2}}{P} ( 1 + {\sum }_{i = 1 … P} {{x}_{{v}_{i}} ( {x}^{T} x )}^{- 1} {{x}_{{v}_{i}}}^{T} )$$

In this example, we can use the residual error from training on subjects without the variant, e=ŷ−y, to estimate σ_{γ}²:

$$E \{ {ee}^{T} \} = E \{ ( \hat{y} - y ) {( \hat{y} - y )}^{T} \} = E \{ ( x \hat{β} - y ) {( … )}^{T} \} = E \{ ( {x ( {x}^{T} x )}^{- 1} {x}^{T} ( x β + γ ) - ( x β + γ ) ) {( … )}^{T} \} = E \{ ( {x ( {x}^{T} x )}^{- 1} {x}^{T} - I ) {γγ}^{T} ( {x ( {x}^{T} x )}^{- 1} {x}^{T} - I ) \} = {{σ}_{γ}}^{2} ( I - {x ( {x}^{T} x )}^{- 1} {x}^{T} )$$

But e^{T}e=Σ_{i=1 . . . N} Diagᵢ(ee^{T}) hence

$$E \{ SSE \} = E \{ {e}^{T} e \} = {\sum }_{1 = 1 … N} {Diag}_{i} ( {{σ}_{γ}}^{2} ( I - {x ( {x}^{T} x )}^{- 1} {x}^{T} ) ) = {{σ}_{γ}}^{2} N - {{σ}_{γ}}^{2} {\sum }_{i = 1 … N} {Diag}_{i} ( {x ( {x}^{T} x )}^{- 1} {x}^{T} ) = {{σ}_{γ}}^{2} ( N - M - 1 )$$

It can be shown that Σ_{i=1 . . . N} Diagᵢ(x(x^{T}x)⁻¹x^{T}) is M+1 where x ϵ [symbol] ^{N×(M+1)}. ρ_{γ}² can be estimated in two ways:

$${{s}_{γ}}^{2} = \frac{{e}^{T} e}{N - M - 1} , \\ {{\hat{σ}}_{γ}}^{2} = \frac{{e}^{T} e}{N}$$

where s_{γ}² is the unbiased estimate of σ_{γ}² and accounts for the M+1 degrees of freedom lost in the regression solution and is typically used; whereas \hat{σ}_{γ}² is the maximum likelihood estimate of σ_{γ}² and is biased, but converges to the same as s_{γ}² for large N.
Replacing s_{γ}² for σ_{γ}², the estimated MSEᵥ may now be computed

$$E \{ {MSE}_{v} \} = \frac{{{s}_{γ}}^{2}}{P} ( 1 + {\sum }_{i = 1 … P} {{x}_{{v}_{i}} ( {x}^{T} x )}^{- 1} {{x}_{{v}_{i}}}^{T} ) = \frac{{e}^{T} e}{P ( N - M - 1 )} ( 1 + {\sum }_{i = 1 … P} {{x}_{{v}_{i}} ( {x}^{T} x )}^{- 1} {{x}_{{v}_{i}}}^{T} )$$

A F-test statistic can be created as follows

$${F}_{v} = \frac{E \{ {MSE}_{v} \}}{{MSE}_{v}} = \frac{\frac{{e}^{T} e}{( N - M - 1 ) P} ( {\sum }_{i = 1 … P} {{x}_{{v}_{i}} ( {x}^{T} x )}^{- 1} {{x}_{{v}_{i}}}^{T} + 1 )}{\frac{{{e}_{v}}^{T} {e}_{v}}{P}}$$

Under the null hypothesis, Fᵥ satisfies an F-distribution with N−M−1 and P degrees of freedom, respectively. One-sided and two-sided p-values can be computed:

p_{1-sided}=f_{cumF}(Fᵥ,N−M−1,P),p_{2-sided}=2×f_{cumF}(Fᵥ,N−M−1,P)

where it is assumed that Fᵥ<1 since the subjects with the variant are assumed to fit the model less well than the subjects that the model was trained on. f_{cumF}(Fᵥ, N−M−1, P) is the integral of the F-distribution of N−M−1 and P degrees of freedom from 0 to Fᵥ. In the case that Fᵥ≥1,

$${p}_{1 - sided} = {f}_{cumF} ( \frac{1}{{F}_{v}} , P , N - M - 1 ) , \\ {p}_{2 - Sided} = 2 \times {f}_{cumF} ( \frac{1}{{F}_{v}} , P , N - M - 1 )$$

Arguably, only the 1-sided p-value is meaningful since the model failure will generate MSEᵥ larger than expected, not smaller; however, the 2-sided value is more conservative.

### Implementation Systems

The methods described here can be implemented on a variety of systems. For instance, in some aspects the system for identifying gene networks includes one or more processors coupled to a memory. The methods can be implemented using code and data stored and executed on one or more electronic devices. Such electronic devices can store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals).

The memory can be loaded with computer instructions to train the classifier to identify a gene network. In some aspects, the system is implemented on a computer, such as a personal computer, a portable computer, a workstation, a computer terminal, a network computer, a supercomputer, a massively parallel computing platform, a television, a mainframe, a server farm, a widely-distributed set of loosely networked computers, or any other data processing system or user device.

The methods may be performed by processing logic that comprises hardware (e.g. circuitry, dedicated logic, etc.), firmware, software (e.g., embodied on a non-transitory computer readable medium), or a combination of both. Operations described may be performed in any sequential order or in parallel.

Generally, a processor can receive instructions and data from a read only memory or a random access memory or both. A computer generally contains a processor that can perform actions in accordance with instructions and one or more memory devices for storing instructions and data. Generally, a computer will also include, or be operatively coupled to receive data from or transfer data to, or both, one or more mass storage devices for storing data, e.g., magnetic disks, magneto optical disks, optical disks, or solid state drives. However, a computer need not have such devices. Moreover, a computer can be embedded in another device, e.g., a smart phone, a mobile audio or media player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including, by way of example, semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

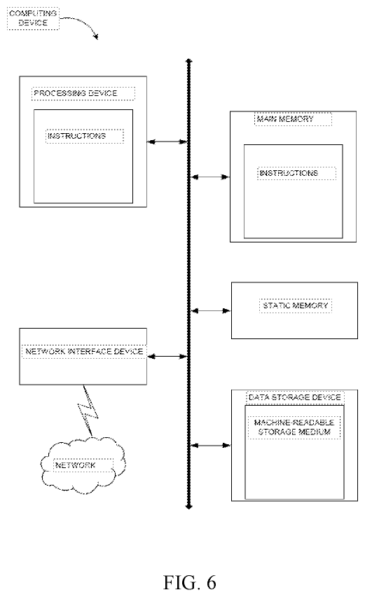

An exemplary implementation system is set forth in FIG. 6. Such a system can be used to perform one or more of the operations described here. The computing device may be connected to other computing devices in a LAN, an intranet, an extranet, and/or the Internet. The computing device may operate in the capacity of a server machine in client-server network environment or in the capacity of a client in a peer-to-peer network environment.

### Diagnosis and Treatment

In some aspects, a subject (e.g., a human subject) is diagnosed as having a condition or disease, or being at risk of having the condition or disease, based on the identified dysregulation of one or more gene networks caused by particular genes or gene variants. In some aspects, a subject (e.g., a human subject) is diagnosed as having a condition or disease, or being at risk of having the condition or disease, based on a perturbed state of a gene network classified by a classification model. For instance, in some aspects a subject having the gene of interest (e.g., a variant in the gene of interest), and/or a particular expression level (e.g., an abnormal expression level) of the gene of interest, is diagnosed as having the condition or disease. In some aspects, a subject having particular expression levels associated with the gene network, is diagnosed as having the condition or disease.

Some aspects comprise identifying a genetic variant in a sample taken from a subject; obtaining gene expression data from the subject; applying the trained model to the gene expression data; and determining whether the genetic variant is pathogenic. In some aspects, the gene expression data is mRNA and/or protein expression data. The gene expression data can be obtained by any known methods, such as RNA Seq or protein analysis. In some aspects, the gene expression data is from a single tissue or biological fluid sample. In some aspects the gene expression data is from a plurality of tissue and/or biological fluid samples from the subject. In some aspects, the gene expression data is combined with gene expression data from one or more tissue and/or biological samples from one or more additional subjects. The trained model can be applied to the combined data to determine whether a genetic variant is pathogenic.

Some aspects comprise treating a subject determined to have a condition or disease. The term “treat” is used herein to characterize a method or process that is aimed at (1) delaying or preventing the onset or progression of a disease or condition; (2) slowing down or stopping the progression, aggravation, or deterioration of the symptoms of the disease or condition; (3) ameliorating the symptoms of the disease or condition; or (4) curing the disease or condition. A treatment may be administered after initiation of the disease or condition. Alternatively, a treatment may be administered prior to the onset of the disease or condition, for a prophylactic or preventive action. In this case, the term “prevention” is used. In some aspects the treatment comprises administering a drug product listed in the most recent version of the FDA's Orange Book, which is herein incorporated by reference in its entirety. Exemplary treatments are also described PHYSICIANS' DESK REFERENCE (PRD Network 71st ed. 2016); and THE MERCK MANUAL OF DIAGNOSIS AND THERAPY (Merck 20th ed. 2018), each of which are herein incorporated by reference in their entirety.

The following examples are provided to illustrate the invention, but it should be understood that the invention is not limited to the specific conditions or details of these examples.

### EXAMPLES

### Example 1

### Tuberous Sclerosis Gene Network Identification

The approach outlined above was used to identify a gene network for Tuberous sclerosis (TSC), which is an autosomal dominant disorder caused by mutations in the TSC1 or TSC2 genes. The symptoms of TSC involves benign tumors growing in the brain and on other vital organs such as the kidneys, heart, liver, eyes, lungs, and skin. Without being bound by theory, the signaling pathway involving TSC1 and TSC2 is believed to be understood. Hoogeveen-Westerveld et al. (Functional Assessment of TSC2 Variants Identied in Individuals with Tuberous. Human Mutation. Volume 34, Issue 1. First published 17 Aug. 2012). The TSC1 and TSC2 gene products, TSC1 (hamartin) and TSC2 (tuberin), form a complex TSC1-TSC2 for which both genes need to be functioning correctly to be stable and have the right activity. Amino acids 1-900 in TSC2 control the interaction with TSC1; while amino acids 1,525-1,770 in TSC2 form protein (GAP) domain which actives GTPase. Specifically, the TSC1TSC2 complex acts on the GTPase Ras homologue expressed in brain (RHEB) to inhibit RHEBGTP-dependent stimulation of the mammalian target of rapamycin (mTOR) complex 1 (TORC1). Consequently, reduction or inactivation of the TSC1TSC2 complex results in increased TORC1 activation, which results in increased phosphorylation of the downstream targets of TORC1 including p70 S6 kinase (S6K), ribosomal protein S6, and 4E-BP1. Consequently, to determine whether TSC1 and TSC2 variants are disease-causing, the activity of RHEB GTPase, and the phosphorylation status of S6K, S6, and 4E-BP1, can be measured.

A functional study was conducted on several variants identified by Hoogeveen-Westerveld in which a standardized immunoblot assay was used to compare variants in TSC2 to wild-type TSC2 and to the known pathogenic variant of TSC2 R611Q. In at least 4 independent transfection experiments for each variant evaluated, separate expression constructs encoding TSC2 with the relevant variant, TSC1, and S6K were cotransfected into HEK 293T cells. Twenty-four hours after transfection, the cells were harvested, lysed, and subjected to immunoblotting to assess levels of TSC2, TSC1, and S6K expression, as well as levels of S6K-T389 phosphorylation. The integrated intensities of the bands on the immunoblots corresponding to TSC2, TSC1, total S6K, and T389-phosphorylated S6K (T389) were determined for each variant, relative to the signals for the wild-type TSC1TSC2 complex. The mean values for the TSC2, TSC1, and S6K signals, and the mean ratios of T389-phosphorylated S6K to total S6K (T389/S6K ratio) were measured.

One mutation of TSC2 gene, involving a change in the amino acid at position 1679 from “E” to “K” (E1679K) has been classified as “benign”, “likely benign” or “variant of unknown significance” by a number of established diagnostic laboratories as recorded in Clinvar. This is largely because in the canonical functional study Hoogeveen-Westerveld, the protein levels for TSC2, TSC1, and the ratio T389/S6K were not found to be statistically different from the wildtype expression levels. This, combined with the fact that the variant occurs at a relatively high frequency in certain population groups—as high as 0.0465% or 1 in 2151—resulted in the variant being classified as “probably neutral” in the study. The variant E1679K is known to occur in GAP region which activates GTPase. Nonetheless, a subject with the E1679 mutation was determined to have certain mild symptoms associated with Tuberous Sclerosis, such as angiolipomas, angiomyolipomas, hemangiomas, and a kidney cyst.

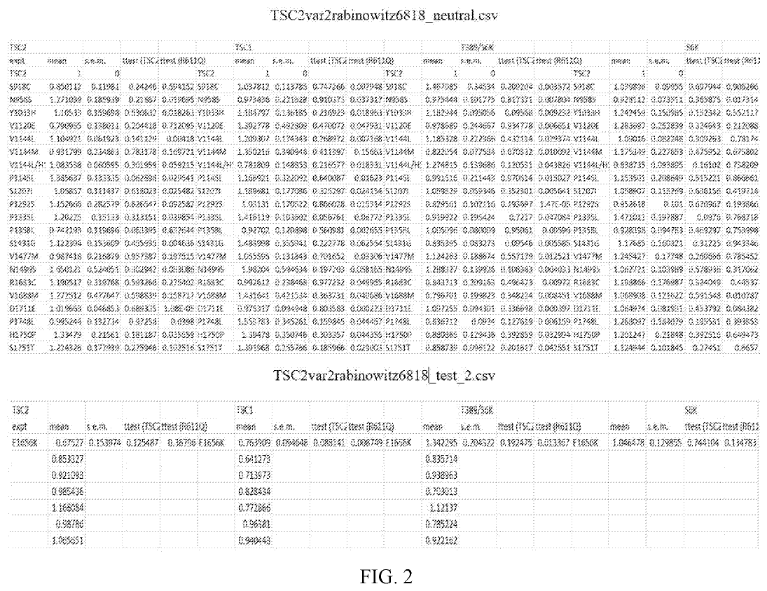

Since TSC2 stabilizes TSC1 in the TSC1-TSC2 complex, a deterministic reduction in expression of TSC2 from the mutation are assumed to cause a commensurate reduction in TSC1. Indeed, reduced expression levels of TSC1 and TSC2 are observed to be reduced by similar amounts. With this in mind, data from a protein functional study and the knowledge of signaling pathways was combined using a MATLAB code to determine whether the reduced expression levels of TSC1 and TSC2 and the increased ratio T389/S6K, in fact indicate a significant effect of the E1679K mutation on TSC2. Aspects of the the MATLAB code used for this example is provided in Appendix A (CSV data files are set forth in FIG. 2):

When the method was applied to all three protein measurements for TSC2, TSC1 and T389/S6K, which checks whether the TSC2 gene is correctly functioning to modulate downstream TSC1 and T389/S6K, as well as whether TSC2 is correctly functioning to maintain it's own expression level, P_{1-sided}=0.1704. In this test, (i) TSC1 is modeled in terms of TSC2, (ii) TSC2 is modelled only in terms of its mean signal level for subjects without the mutation, and (iii)T389/S6K is modelled in terms of TSC1 and TSC2, where TSC1 is included to control for that cofactor. If each of these effects are considered in isolation, one-sided p-values for effects for (i), (ii), and (iii) are 0.1388, 0.4152, and 0.2560, respectively. If the downstream modulation effects of TSC2 on TSC1 and T389/S6K are separated out, we obtain p_{1-sided}=0.1662. These results indicate we cannot state that the gene not still correctly functioning since it is modulating downstream genes and maintaining it's own level in the test data, roughly as expected based on the training data.

### Example 2

### Considering Mean Signal Levels to Analyze Gene Expression Level Rather than Gene Function

A model was created to analyze whether the mean expression level of the TSC2 gene is changed by considering the mean levels of TSC1 and T389/S6K. This does not check whether TSC2 is correctly modulating TSC1 and T389/S6K, but rather assumes that it is modulating the downstream genes as it does when correctly functioning. With that assumption, the approach checks the downstream gene level to determine if that corroborates a changed level of TSC2. In this case, the model for a mutated gene and downstream gene can be described as:

x=1βₓ+∝;y=1β_{y}+γ;x,y,∝,γϵR^{N×1};βₓ,β_{y}ϵR^{1×1}

where 1 is the column matrix of all 1's. Similarly, we may represent the data for the mutated subjects

xᵥ=1βₓᵥ+∝ᵥ;yᵥ=1β_{yv}+γᵥ;xᵥ,yᵥ,∝ᵥ,γᵥϵR^{N×1};βₓᵥ,β_{yv}ϵR^{1×1}

All the noise distributions may be considered normal and i.i.d. In the null hypothesis that the expression level of gene x is unaffected, the means are the same: βₓ=βₓᵥ. If downstream gene y mean level depends on x, then β_{y},β_{yv} will be functions of βₓ,βₛᵥ, consequently in the NULL hypothesis, β_{y}=β_{yv}.
Using the least squares approach, we can solve for \hat{β}ₓ=(1^{T}1)⁻¹1^{T}x,\hat{β}_{y}=(1^{T}1)⁻¹1^{T}y which are simply the sample means \hat{β}ₓ=x,\hat{β}_{y}=y. The same approach outlined above was used to compute the p-values for these models, namely

$$E \{ {SSE}_{{y}_{v}} \} = E \{ {( {\hat{y}}_{v} - {y}_{v} )}^{T} ( {\hat{y}}_{v} - {y}_{v} ) \} = E \{ {( 1 \underline{y} - {y}_{v} )}^{T} ( 1 \underline{y} - {y}_{v} ) \} = {{N σ}_{{γ}_{v}}}^{2} + {{σ}_{γ}}^{2}$$

$$E \{ {SSE}_{{x}_{v}} \} = {{Nσ}_{{α}_{v}}}^{2} + {{σ}_{α}}^{2}$$

$${MSE}_{{y}_{v}} = \frac{1}{N - 1} {SSE}_{{y}_{v}} , \\ {MSE}_{{x}_{v}} = \frac{1}{N - 1} {SSE}_{{x}_{v}}$$

Since 1 degree of freedom is given up in computing the mean, this approach was applied to generate p-values based on an F-statistic. When this approach was applied to the signals TSC2, TSC1, and T389/S6K in combination, the 1-sided p-value was 0.0433. When applied to each of the gene signals separately, the 1-sided p-value were respectively 0.4152, 0.0538 and 0.3010. This indicates that the variant is likely affecting the mean expression level of TSC2 which in turn is effecting the mean expression level for TSC1 and T389/S6K. The statistical significance is higher when the signals are combined. Aspects of the MATALAB code used for this example are provide in Appendix A.

Another approach which may be favorable for the mean analysis above is to generate a Student T-statistic, rather than an F-Statistic based on MSE. In this approach, a test statistic was calculated based on the difference of the means.

eₘₓ=xᵥ−x,e_{my}=yᵥ−y

The variance of each of these is given by:

$$Var ( {e}_{mx} ) = E \{ {( {e}_{mx} - E ( {e}_{mx} ) )}^{2} \} = \frac{{{σ}_{α}}^{2}}{N} + \frac{{{σ}_{{α}_{v}}}^{2}}{{N}_{v}}$$

$$Var ( {e}_{my} ) = E \{ {( {e}_{my} - E ( {e}_{my} ) )}^{2} \} = \frac{{{σ}_{γ}}^{2}}{N} + \frac{{{σ}_{{γ}_{v}}}^{2}}{{N}_{v}}$$

The noise variances was estimated based on the mean-squared errors

$${{σ}_{α}}^{2} \approx {MSE}_{α} = \frac{{( x - \underline{x} )}^{T} ( x - \underline{x} )}{N - 1} , \\ {{σ}_{γ}}^{2} \approx {MSE}_{γ} = \frac{{( y - \underline{y} )}^{T} ( y - \underline{y} )}{N - 1} , \\ {{σ}_{{α}_{v}}}^{2} \approx {MSE}_{{∝}_{v}} = \frac{{( {x}_{v} - {\underline{x}}_{v} )}^{T} ( {x}_{v} - {\underline{x}}_{v} )}{{N}_{v} - 1} , \\ {{σ}_{{γ}_{v}}}^{2} \approx {MSE}_{{γ}_{v}} = \frac{{( {y}_{v} - {\underline{y}}_{v} )}^{T} ( {y}_{v} - {\underline{y}}_{v} )}{{N}_{v} - 1}$$

A t-statistic for each of the genes was created:

$${t}_{x} = \frac{{e}_{mx}}{\frac{{MSE}_{α}}{N} + \frac{{MSE}_{{α}_{v}}}{{N}_{v}}} , \\ {t}_{y} = \frac{{e}_{my}}{\frac{{MSE}_{γ}}{N} + \frac{{MSE}_{{γ}_{v}}}{{N}_{v}}}$$

p-values for each of these genes separately was computed as follows:

p_{x,1-sided}=1−f_{cumT}(|tₓ|,N+Nᵥ−2),p_{x,2-sided}=2−2×f_{cumT}(|tₓ|,N+Nᵥ−2)

p_{y,1-sided}=1−f_{cumT}(|t_{y}|,N+Nᵥ−2),p_{2-sided}=2−2×f_{cumT}(|tₓ|,N+Nᵥ−2)

where N+Nᵥ−2 represents the degrees of freedom from summing N and Nᵥ normal random variables and losing 2 degrees by computing the means; and F_{T}(|tₓ|,N+Nᵥ−2) represent cumulative density function of Student-T distribution with N+Nᵥ−2 degrees of freedom, at |tₓ|. Using this approach applied separately to each of TSC2, TSC1 and T389/S6K, in aspects of the MATLAB code set forth in Appendix A, 2-sided p-values respectively of 0.0372, 3.9058e-6 and 0.5036, were obtained.

Note that, particularly if K genes are separately analyzed, one can reduce the threshold p-value by roughly K, as per Bonferroni correction, (reference: Goeman, Jelle J.; Solari, Aldo (2014). “Multiple Hypothesis Testing in Genomics”. Statistics in Medicine. 33 (11): 1946-1978. doi:10.1002/sim.6082. PMID 24399688). For example, for K=3, if the target p-value is typically 5%, one would only flag a statistical anomaly on the example above at a p-value of below 5%/3=1.67%.

### Example 4

### Integrating over Multi-Dimensional Probability Distributions

The challenge with the t-statistic approach is that it is difficult to combine the test statistics across different genes, whereas in the F-test one could simply sum the SSE from multiple genes. One way of addressing this, so that signals can be combined across many genes, is to consider the integration over a multi-dimensional probability distribution where each gene represents one dimension. Consider in the general case variables T₁ . . . T_{K} which have joint probability distribution function f_{T}_{₁} _{^{. . . T}}_{K} (t₁, . . . t_{K}). If one considers the mutated gene and all downstream genes, for example, K=1+D, the cumulative distribution function for these variables is given by

f_{cum,T}_{₁} _{^{. . . T}}_{K}(t_{1,} . . . t_{K})=∫_{-∞}^{t}^{₁} . . . ∫_{-∞}^{t}^{_{N}}f_{T}_{₁} _{^{. . . T}}ₖ(t_{1,} . . . t_{K})dt₁ . . . dtₖ

The p-value for a series of values t . . . t_{K} is given by pₜ_{₁} _{^{. . . t}}ₖ=1−2^{K}F_{T}_{₁} _{^{. . . T}}ₖ(t_{1,} . . . t_{K}) where all p-values computed from a probability density function of multiple dimensions are one-sided and the 2^{K}multiple accounts for the fact that we are integrating in only one quadrant in two dimensions, or only over the space of the positive axes in multiple dimensions. Since the integral described above may be computationally complex, one approach is to combine all of the test statistics t_{1,} . . . t_{K} in each dimension into a single radial test statistic as follows:

r=\sqrt{Σ_{k=1 . . . K}^{t}^{ₖ}²},

where r is now the radius of the multi-dimensional sphere that encompasses the multi-dimensional rectangle defined by t . . . t_{K} hence it is a more conservative test statistic. The volume of the sphere of this radius in K dimensional space can be computed:

$$V ( k . r ) = \frac{2 {π}^{\frac{K}{2}}}{gamma ( \frac{K}{2} )} \frac{{r}^{K}}{K}$$

where gamma is Euclid's gamma function. The length of the side of a cuboid that has the same volume as the spheroid in this K-dimensional space was found as follows:

$$l = {r}^{\frac{1}{K}}$$

The p-values were then computed with a more computationally tractable and more conservative integration over a multi-dimensional probability distribution

$${p}_{{t}_{1} … {t}_{K}} \approx {\int }_{- \frac{l}{2}}^{\frac{l}{2}} … {\int }_{- \frac{l}{2}}^{\frac{l}{2}} {f}_{{T}_{1} … {T}_{K}} ( {t}_{1} … {t}_{K} ) {dt}_{1} … {dt}_{K}$$

This approach implemented for the multi-dimensional Student-T and normal distributions in aspects of the MATLAB code set forth in Appendix A was used to combine the T-test statistics of TSC1, TSC2 and T389/S6K. 1-sided p-value of 1.4398e-005 were obtained. Notwithstanding the possibility of normal noise assumptions, this indicates the likelihood that the mutation is disrupting gene expression levels.

This same method can be implemented with a range of gene expression databases. In order to determine whether this variant was causative, data from subjects that had their RNA or protein expression levels measured for TSC1, TSC2 and T389/S6K, were used. For each, the RNA expression levels was analyzed for subjects that do and do not have the mutation using the methods outlined above. For a mutation that has roughly 1/2000 incidence, with a gene expression database of 100,000 cases one would have roughly 50 individuals with the variant. That could be enough to average out the noise, effects of lifestyle, timing and an array of other phenotypes to see if this mutation affected gene function.

### Example 5

### Using the Model of Gene Signaling Pathways or Gene Expression as a Functional Assay

Many techniques have emerged using editing technology such a CRISPR/Cas9 or CRISPR with other Cas-like cutting enzymes, or other editing methods, to genetically perturb the DNA of cells and see if a phenotype is created by various mutations. For instance, this was successfully done for BRCA1 mutations where an effective functional assay was available to show the loss of function of the BRCA1 gene. One challenge of these approaches is that the functional assay can be difficult to determine or measure for particular genes. Methods described herewhich can use models of gene expression or gene signaling, to determine whether a cell is disrupted by a particular mutation address this problem. Many methods exist for perturbing the DNA of cells and measuring their gene expression in the form of proteins or RNA, such as by RNA-Seq. See for example Dixit et al (“Perturb-seq: Dissecting molecular circuits with scalable single cell RNA profiling of pooled genetic screens.”, Cell. 2016 Dec. 15; 167(7): 1853-1866.e17), which is herein incorporated by reference in its entirety. In this approach, pooled “single guide RNA” (sgRNA) libraries are combined with cells in droplets. Each droplet is designed to take up a single cell and 0, 1, 2, 3 or more sgRNAs targeting particular genetic loci. Each droplet is also barcoded so that barcoded messenger RNA (mRNA) libraries can be created and associated with each cell. In approaches such as this, one can identify the DNA edits for each cell either by sequencing the RNA from the single cell to determine what variants are present, or by including along with the cell barcodes additional barcodes that are associated with each targeted locus, or each sgRNA.

The same techniques described above can be used to determine whether a particular perturbation is causing a change in the cell phenotype. Namely, a model can be trained by considering N cells that do not have DNA carrying any known deleterious mutations, and test by considering P cells that do have a particular mutation or combination of mutations. Similar to the approach above:

y=xβ+γlx=μ+∝;y,γϵR^{N×1};βϵR^{(M+1)×1};xϵR^{N×(M+1)}

yᵥ=xᵥβᵥ+γᵥ;xᵥ=μᵥ+αᵥ;yᵥ,γᵥϵR^{P×1};βᵥϵR^{(M+1)×1};xαᵥϵR^{P×(M+1)}

\hat{β}=argmin_{β}(y−xβ)^{T}(y−xβ)=(x^{T}x)⁻¹x^{T}y;ŷx\hat{β};ŷᵥ=xᵥ\hat{β}

e=ŷ−y;eᵥ=ŷᵥ−yᵥ

The F-test statistic can be computed:

$${F}_{v} = \frac{E \{ {MSE}_{v} \}}{{MSE}_{v}} = \frac{\frac{{e}^{T} e}{( N - M - 1 ) P} ( {\sum }_{i = 1 … P} {{x}_{{v}_{i}} ( {x}^{T} x )}^{- 1} {{x}_{{v}_{i}}}^{T} + 1 )}{\frac{{{e}_{v}}^{T} {e}_{v}}{P}}$$

$${F}_{v} < 1 \text{:} {p}_{1 - sided} = {f}_{cumF} ( {F}_{v} , N - M - 1 , P ) , \\ {p}_{2 - sided} = 2 \times {f}_{cumF} ( {F}_{v} , N - M - 1 , P )$$

$${F}_{v} \geq 1 \text{:} {p}_{1 - sided} = {f}_{cumF} ( {F}_{v} , N - M - 1 , P ) , \\ {p}_{2 - sided} = 2 \times {f}_{cumF} ( {F}_{v} , N - M - 1 , P )$$

Namely, using the single hypothesis rejection test, one can reject the null hypothesis H₀ that the variant does not affect cell function if the p-value is less than some threshold. Rather than using a single-hypothesis rejection test, one can also create a maximum-likelihood test where multiple different models \hat{β} are considered, and the model that is the best fit for yᵥ is identified. For example, a model of \hat{β} can be created based on cells that do not have any known pathogenic mutations and a different model of \hat{β} based on cells that are known to carry a pathogenic mutation. Then, one can check to see which RNA expression pattern the expression signals from the perturbed test cells yᵥ match better, based on the approaches described above. One rejects the hypothesis with the lowest p-value, namely one rejects hypothesis H₀ that the variant is benign if the expression signals best match the model built from cells with pathogenic mutations, and one rejects hypothesis H₁ that the variant is pathogenic if the expression signals best match the model built from cells without pathogenic mutations.

One can also extend this method to a Bayesian framework where a test statistic is combined with a prior probability of a variant being pathogenic. For example, the probability of each hypothesis given the tests statistic is:

$$P ( {H}_{0} \vert {F}_{v} ) = \frac{P ( {F}_{v} \vert {H}_{0} ) P ( {H}_{0} )}{P ( {F}_{v} )} ;$$

$$P ( {H}_{1} \vert {F}_{v} ) = \frac{P ( {F}_{v} \vert {H}_{1} ) P ( {H}_{1} )}{P ( {F}_{v} )}$$

Consequently, one selects H₀ if P(Fᵥ|H₀)P(H₀)>P(Fᵥ|H₁)P(H₁) and H₁ otherwise. The method may also be extended to having multiple hypotheses, where each matches a particular state of the cell, for example milder or more severe phenotypes. In this way, the RNA or protein expression levels of single cells can be used as a highly scalable laboratory assay for testing whether particular variants or combinations of variants are deleterious. This can be applied over a wide range of diagnostic tests to differentiate variants of unknown significance from benign or pathogenic variants, including carrier testing, cancer susceptibility testing, cancer somatic testing, whole exome or whole genome testing and a vast array of other genetic screening or diagnostic panels.

### Example 6

### Determining Causative Variants from Gene Expression Networks

It is assumed that individuals that have pathogenic variants have a transcriptomic signature that can be used to better classify variants of unknown significance and determine if they are pathogenic or benign. The hypothesis explored in this example is that the genotype—phenotype relationships in the GTEx database can be used as proof of concept method to determine if changes in the transcript abundance of the network of genes related to the gene of interest can be detected in a way that can improve classification of VUS in the gene of interest. The methods in this example are purely illustrative and can be modified in a wide variety of ways using different genes, difference databases, different measures of expression, different normalization techniques, different statistical analysis techniques and different measures of efficacy of the models, without changing the essential concept.

GTEx expression data was used along with the rare variants classified using Clinvar pathogenic and benign variants to identify gene networks, as shown in FIG. 1B. FIG. 1A is a demonstration of a network of M Midstream Genes Affecting D Downstream Genes, and FIG. 1B represents an exemplary classifier generation method for determining whether a variant of unknown susceptibility should be classified as a benign or pathogenic variant. GTEx Genotype and Phenotype datasets include 979 individuals that were genotyped using WES and tissue sampled from up to 54 body locations per individual post-mortem. Many individuals had pathogenic and benign variants, and some have both on a per gene basis. For each gene, we selected a subset of individuals carrying pathogenic, benign, or both variants. Those individuals were used to select a subset of tissues that have expression data (FIG. 1B). To determine the set of pathogenic variants that are considered for the classification, we filtered the Clinvar database to pathogenic variants that follow the criteria (i) in an autosomal dominant gene, (ii) there exist many lines of evidence that the variant is pathogenic, (iii) there are no contradicting submissions about the classification, i.e. all submissions are pathogenic. To generate the initial set of pathogenic/benign labels, bedtools isec was used to find overlaps between individuals with pathogenic/benign variants from Clinvar and GTEx V8 WES individuals (inner join). Joining the two datasets yields 502 pathogenic variants in 326 genes, 21286 benign variants in 2186 genes, and 292 genes with both pathogenic and benign variants; individuals without benign variants were treated as a control. Those data were imported to an ipython notebook and analysis was started from that point.

To identify a list of genes of interest, all variants counts per gene, per individual were aggregated and sorted to find genes that had the largest number of individuals with pathogenic variants (Table 1). The subsequent analysis demonstrates the classifier trained on the gene PRSS1

TABLE 1
Data from GTEx and Clinvar was accumulated per gene.

| gene_name | num_benign_indiv | num_path_indiv | overlap_ben_path |
|---|---|---|---|
| PRSSI* | 919 | 703 | 688 |
| SERPINAI | 735 | 97 | 63 |
| BTD* | 240 | 61 | 13 |
| PRRT2 | 23 | 59 | 1 |
| BCHE* | 37 | 40 | 2 |
| TYR | 534 | 15 | 4 |
| ALDOB | 6 | 15 | 0 |
| CEP290 | 972 | 13 | 12 |
| PMM2 | 642 | 13 | 5 |
| GAA | 923 | 11 | 10 |
| GNRHR | 174 | 10 | 8 |
| PRKN | 456 | 10 | 5 |
| SBDS | 314 | 9 | 6 |
| KIAA0586 | 14 | 8 | 0 |
| CLCN1 | 940 | 8 | 7 |
| HOGA1 | 515 | 8 | 7 |
| SPG7 | 884 | 8 | 6 |
| POLG | 811 | 7 | 6 |
| PKLR | 501 | 7 | 1 |
| ABCA3 | 517 | 7 | 2 |

PRSS1 variants are found in 919 individuals, and pathogenic variants are found in 703 individuals, there were 688 individuals with both pathogenic and benign variants. Pathogenic labels were applied to all individuals that had a pathogenic variant regardless if they have a benign variant. Benign labels were applied to all benign variants that did not have a pathogenic variant, or had no classification at all (including VUS).

The GTEx dataset contains several (1 or more) expression profiles per tissue and several (1 or more) samples per individual. To account for correct sampling of test-train, we split the test train samples so that the testing phase does not evaluate samples from individuals that were in the training phase. The resulting split generated 3 cohorts of samples, each containing a unique list of tissue samples from different people. The number of benign and pathogenic individual samples in each cohort are included in Table 2. For paragraphs 84-87, results will be described for cohort 1, and a comparison of performance on cohorts 2 and 3 are described in paragraph 88.

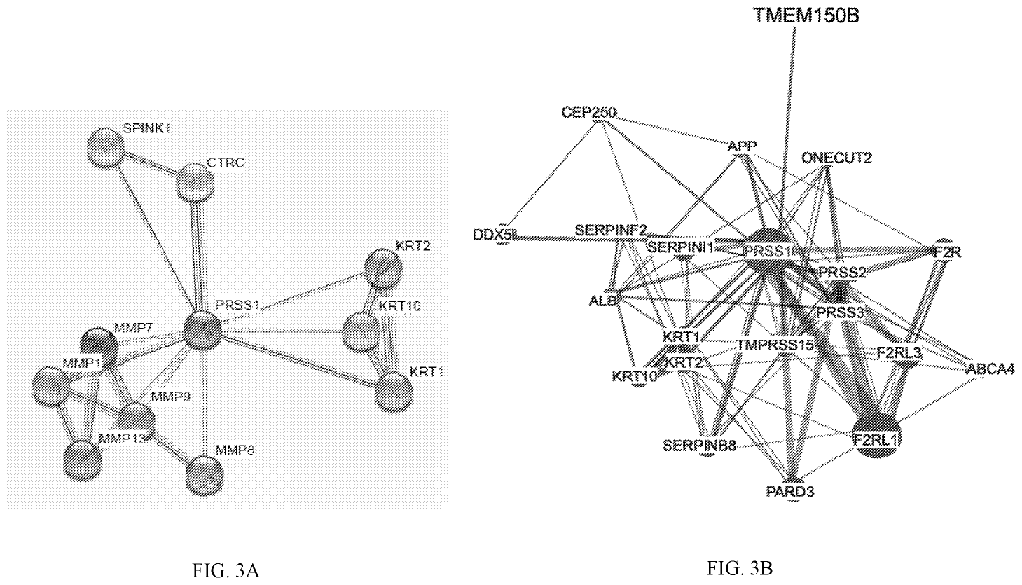

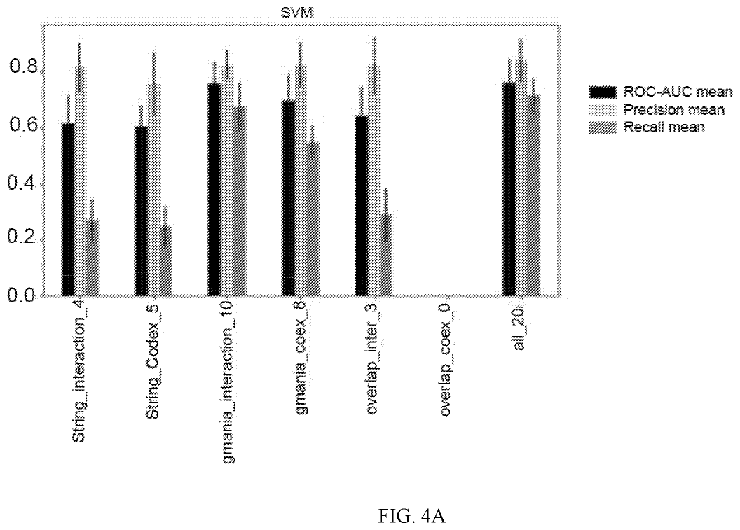

The Gene network feature selection method constructs 6 putative gene-gene networks and one aggregate network from all genes known to interact with PRSS1 (7 network ensembles). To identify genes that interact in a network we queried 2 public databases that aggregate published interactions; GeneMania (citation) and String V11 (citation) (FIG. 3, (A) GeneMania, (B) String V11) . Gene-gene interaction tables were downloaded from websites, tabulated and filtered only for gene-gene interactions that were either ‘physical interaction’ or ‘co-expression’, all other interaction categories were discarded. This yields 2 networks per database. Two additional ensembles were created by taking the overlap of genes in interaction categories between the different databases (physical interaction (GeneMania & String v11). A 7th ensemble was constructed by combining all genes from all putative networks. A list of ensemble and associated genes for PRSS1 is in Table 2. This generates 7 train-test matrixes used for classification each of 382 X #_of_genes_in_ensemble, see table 2 per ensemble size and genes included in the network.

Expression levels in all selected network ensembles were normalized to the geometric mean expression level of the housekeeping genes GAPDH','RPS27′, ‘POM121C’.

TABLE 2
Gene ensembles used for feature selection

| Ensemble name | Genes |
|---|---|
| String | PRSS1′, ′KRT2′, ′KRT10′, ′KRT1′ |
| interaction | |
| String | ′CTRC′, ′MMP1′, ′SPINK1′, ′PRSS1′, ′KRT1′ |
| coexpression | |
| Genemania | ′TMEM150B′, ′SERPINB8′, ′APP′, ′DDX5′, |
| interaction | ′KRT2′, ′SERPINF2′, ′KRT10′, ′KRT1′, ′ALB′, |
| | ′CEP250′ |
| GeneMania | PRSS2′, ′PRSS3′, ′F2R′, ′ABCA4′, ′ONECUT2′, |
| coexpression | ′TMPRSS15′, ′ALB′, ′KRT10′ |
| Overlap | ′KRT2′, ′KRT10′, ′KRT1′ |
| interacion | |
| Overlap | None |
| coexpression | |
| All | ′APP′, ′SERPINF2′, ′SPINK1′, ′PRSS2′, ′KRT1′, |
| | ′KRT10′, ′F2R′, ′ABCA4′, ′CEP250′, ′DDX5′, |
| | ′CTRC′, ′MMP1′, ′SERPINB8′, ′KRT2′, |
| | ′TMEM150B′, ′TMPRSS15′, ′PRSS1′, ′ALB′, |
| | ′ONECUT2′, ′PRSS3′ |

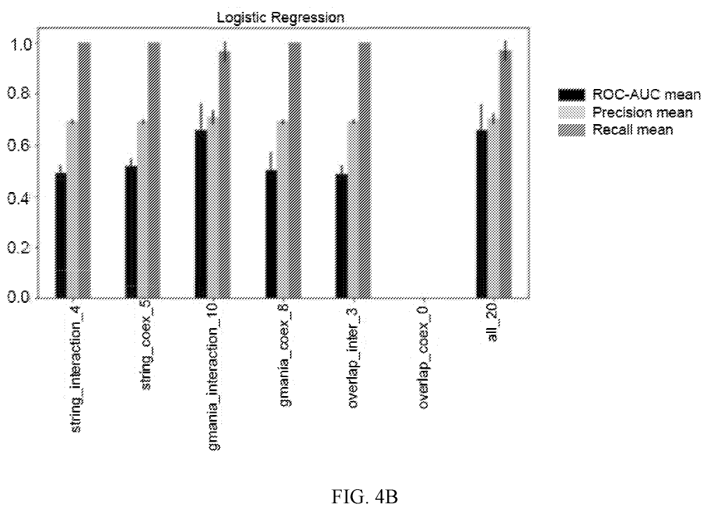

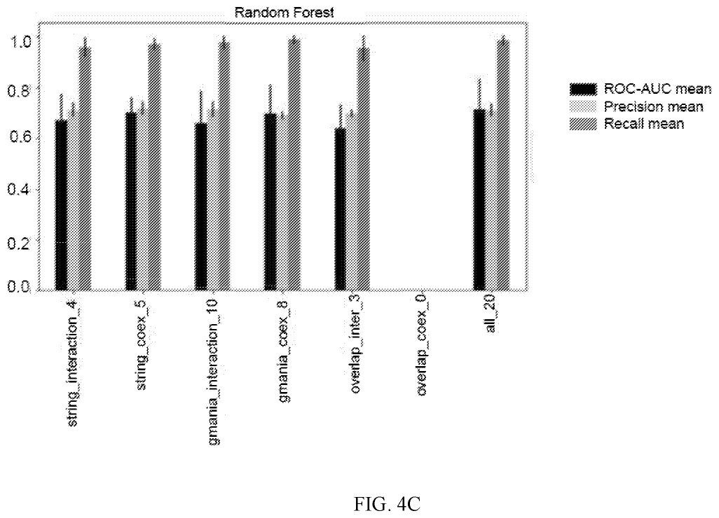

Binary classifiers SVM, logistic regression, and Random Forest were used for training and testing using cross validation technique on the train-test matrix. The cross validation test-train split methodology takes the test-train matrix and splits it into 80:20 samples for training and testing and scores the AUC, precision (PPV, type 1 error) and recall (TPR, type 2 error) of a classifier. To avoid overfitting, cross-validation methodology is used by randomly splitting the data 80:20, for 10 repetitions. The results are set forth in FIG. 4). X axis are the ensembles of genes in each network used for feature selection. Legend of bars; blue: AUC, orange: precision, green: recall. Error bars represent the error due to cross validation and the STD of 10 fold cross validation is present for each bar.

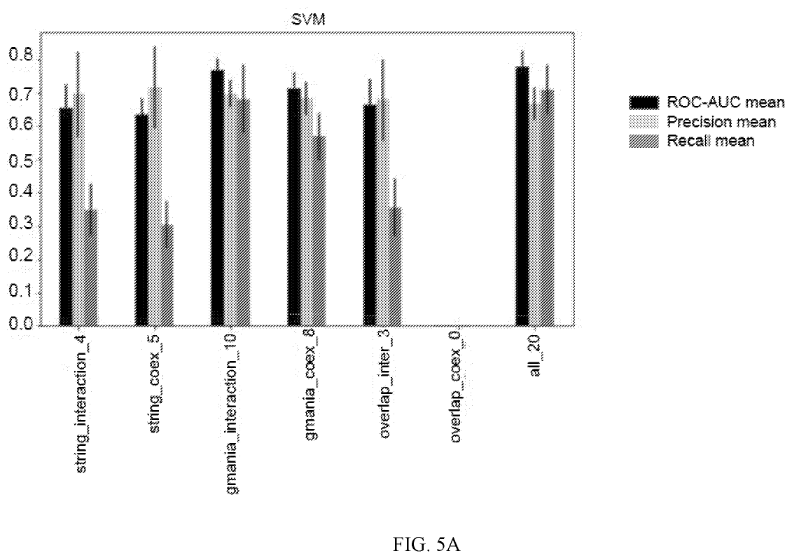

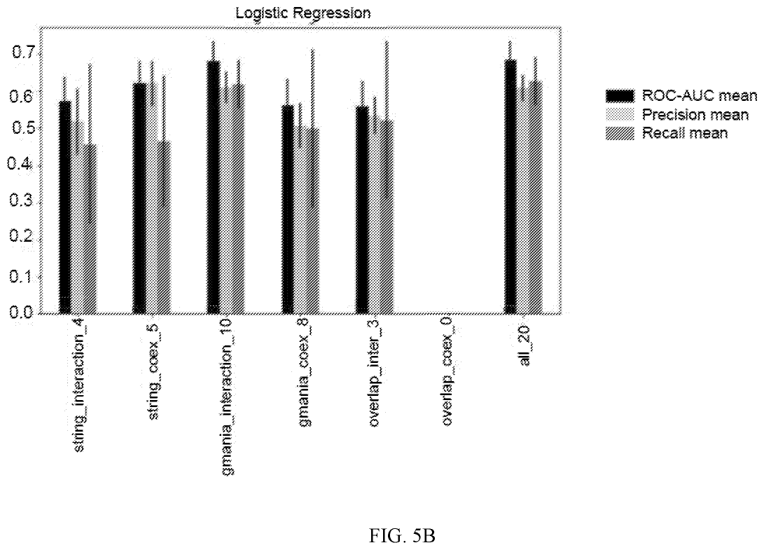

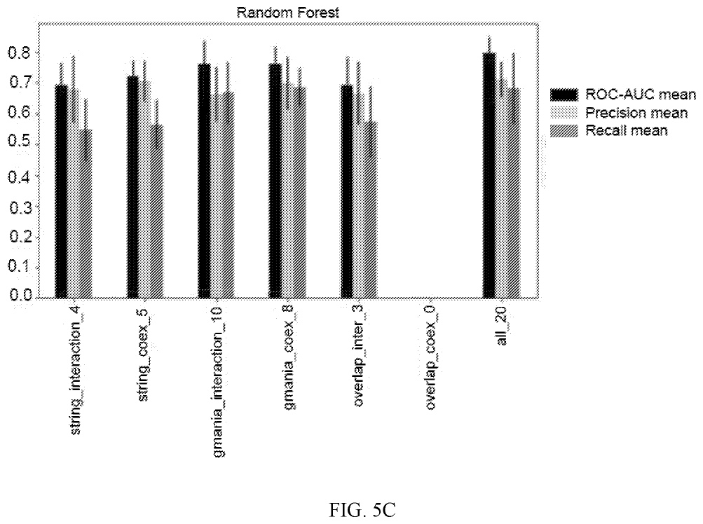

To adjust for the bias of pathogenic and benign variants, expression profiles of benign variants were oversampled to match the total label count of pathogenic variants. The resulting train/test matrix is 528 X #_of_genes_in_ensemble. Results of classification using oversampling are set forth in FIG. 5. X axis are the ensembles of genes in each network used for feature selection. Legend of bars; blue: AUC, orange: precision, green: recall. Error bars represent the error due to cross validation and the STD of 10-fold cross validation is present for each bar.

Training the classification model was done by using a single sample per individual (cohort 1). We wanted to understand if there is biological variance using a different set of transcripts from the same cohort. To address this concern, we trained the classifiers for cohort 2 and 3 on samples that were not used to train the classifier for cohort 1 (SVM and Random Forest). Results are set forth in Table 3:

TABLE 3
comparison of classification amongst different sample cohorts
within the same individual pool.

| | | |
|---|---|---|
| cohort 1 | cohort 2 | cohort 3 |

A

| | | | |
|---|---|---|---|
| gmania_coex_8 | 0.71 +/− 0.04 | 0.76 +/− 0.04 | 0.68 +/− 0.05 |
| gmania_interaction_10 | 0.76 +/− 0.03 | 0.75 +/− 0.04 | 0.74 +/− 0.06 |
| all_20 | 0.77 +/− 0.04 | 0.77 +/− 0.03 | 0.76 +/− 0.07 |

B

| | | | |
|---|---|---|---|
| gmania_coex_8 | 0.75 +/− 0.05 | 0.79 +/− 0.04 | 0.77 +/− 0.043 |
| gmania_interaction_10 | 0.75 +/− 0.07 | 0.81 +/− 0.03 | 0.77 +/− 0.05 |
| all_20 | 0.79 +/− 0.05 | 0.81 +/− 0.04 | 0.79 +/− 0.06 |

Similarity between biological sample cohorts using A) SVM, B) Random Forest classifiers. Results are shown for the networks with the highest ROC.

### Appendix A

### % MATLAB

The invention claimed is:

1. A method of determining the pathogenicity of a genetic variant, the method comprising:
receiving, by a processing device, a first training dataset that includes expression data for a gene of interest and one or more other genes that are up-regulated, down-regulated, or co-expressed with the gene of interest, and/or that interact directly or indirectly with the gene of interest or genes that the gene of interest interacts directly or indirectly with;
receiving a second dataset that includes phenotypic and/or genotypic data associated with the presence, absence, and/or expression levels of the gene of interest and/or the other genes;
training a model on the first and second training datasets to identify a network of genes associated with phenotypic outcomes; and
scoring the pathogenicity of the gene of interest based on the network of genes associated with phenotypic outcomes.

2. The method of claim 1, further comprising receiving a list of genes from a third dataset that includes data associated with known gene-gene, gene-protein and/or protein—protein interaction networks for the gene of interest and/or the other genes, and
training the model using the first, second, and third datasets to identify the network of genes associated with phenotypic outcomes.

3. The method of claim 1 or 2, wherein the second dataset comprises phenotypic data associated with benign other genes and pathogenic other genes.

4. The method of any one of claims 1-3, wherein the training comprises one or both of (i) normalizing and standardizing expression levels of the gene of interest and/or the other genes, and (ii) resampling data in the training samples

5. The method of any one of claims 1-4, wherein one or more of SVM, linear regression, logistic regression, random forest, naive bayes, and adaboost are used to identify the network of genes or gene variants associated with the phenotypic outcomes.

6. The method of any one of claims 1-5, wherein the first dataset and/or the second dataset each independently comprises data from a plurality of datasets.

7. The method of any one of claims 1-6, wherein the first and/or second training datasets independently include data from one or more tissues selected from brain, heart, lung, kidney, liver, muscle, bone, stomach, intestines, esophagus, and skin tissue; and/or one or more of a biological fluids selected from urine, blood, plasma, serum, saliva, semen, sputum, cerebral spinal fluid, mucus, sweat, vitreous liquid, and milk, and wherein a test set may include different sets of tissues and/or biological fluids from the tissues and/or biological fluids used for training.

8. The method of any one of claims 1-7, wherein the gene of interest is associated with an autosomal dominant condition; or
wherein the gene of interest is associated with an autosomal recessive condition; or
wherein the gene of interest is associated with a risk for a non-Mendelian condition.

9. The method of any one of claims 1-8, wherein the first dataset is comprised of mRNA and/or protein expression data associated with the gene of interest and/or the other genes or gene variants.

10. The method of any one of claims 1-9, wherein the gene of interest is a genetic variant of interest.

11. A method of constructing a pathogenicity classifier for a genetic variant, the method comprising training a pathogenicity classifier on a processing device, wherein the method comprises using as input:
(i) a first training dataset that includes expression data for a gene of interest and one or more other genes that are up-regulated, down-regulated, or co-expressed with the gene of interest, and/or that interact directly or indirectly with the gene of interest or with genes that the gene of interest directly or indirectly interacts with; and
(ii) a second training dataset that includes phenotypic and/or genotypic data associated with the presence, absence, and/or expression levels of the other genes; and
wherein the method comprises identifying a network of genes that are up-regulated, down-regulated, or co-expressed with the gene of interest, and/or that interact directly or indirectly with the gene of interest or with genes that the gene of interest directly or indirectly interacts with.

12. The method of claim 11, further comprising using as input a third training sample from a third dataset that includes data associated with known gene-gene or gene-protein interactions for the gene of interest and/or the other genes.

13. A method of determining the pathogenicity of a genetic variant, the method comprising:
performing genotyping, and establishing that there is a variant of unknown significance;
measuring expression levels in one or more samples from one or more subjects with the variant, and applying the trained model of any one of claims 1-10; and
determining the pathogenicity of the gene of interest based on the presence, absence, and/or expression levels of the network of genes.

14. The method of claim 13, where the genotyping comprises performing whole genome sequencing, whole exome sequencing or target panel sequencing.

15. The method of claim 13 or 14, wherein measuring expression levels comprises obtaining mRNA and/or protein expression data associated with the gene of interest and/or the other genes.

16. The method of any one of claims 13-15, wherein the first dataset comprises data from a genetic screen that comprises (i) perturbing the DNA of one or more cells and inserting a variant of known significance (benign or pathogenic), and (ii) validating that that the genetic variant of known significance is classifying correctly using measured expression levels.

17. The method of any one of claims 13-16, wherein the second dataset comprises data from a genetic screen that comprises (i) perturbing the DNA of one or more cells, and (ii) determining the phenotype associated with the one or more perturbed cells.

18. The method of claim 16 or 17, wherein the perturbing is conducted with CRISPR.

19. The method of claim 16 or 17 wherein the measured expression levels comprise levels of mRNA of a network of genes identified in the classification model.

20. The method of any of the preceding claims, further comprising:
identifying a genetic variant in a sample taken from a subject;
obtaining gene expression data from the subject;
applying the trained model to the gene expression data; and
determining whether the genetic variant is pathogenic.

21. The method of claim 20, further comprising obtaining a plurality of tissue and/or biological fluid samples from the subject and performing gene expression analysis on the plurality of tissue and/or biological fluid samples.

22. The method of claim 20 or 21, further comprising obtaining gene expression data from one or more tissue and/or biological samples from one or more additional subjects;
combining the gene expression data from the subject and the one or more additional subjects;
applying the trained model to the combined data; and
determining whether the genetic variant is pathogenic.

23. The method of any one of claims 20-22, wherein the gene expression data comprises mRNA and/or protein expression data.
